(19) United States
(12) Patent Application Publication
(10) Pub. No.: US 2018/0112164 A1
(43) Pub. Date: Apr. 26, 2018

(54) Device for Observation, Imaging and Uninterrupted Culturing of Embryos and Cells

(71) Applicants: Michael D. Cecchi, Longboat Key, FL (US); Timothy Schimmel, Randolph, NJ (US); Jacques Cohen, New York, NY (US); Monica Mezezi, Guelph (CA); Michael Ryan Cecchi, Longboat Key, FL (US)

(72) Inventors: Michael D. Cecchi, Longboat Key, FL (US); Timothy Schimmel, Randolph, NJ (US); Jacques Cohen, New York, NY (US); Monica Mezezi, Guelph (CA); Michael Ryan Cecchi, Longboat Key, FL (US)

(21) Appl. No.: 15/334,953

(22) Filed: Oct. 26, 2016

(51) Int. Cl. C12M 1/00; C12M 3/00; C12M 1/34; G01N 21/84

(52) U.S. Cl. CPC C12M 21/02; C12M 23/22; C12M 23/50; C12M 23/42; G01N 21/84; C12M 31/02; C12M 41/34; C12M 41/14; C12M 41/08; C12M 23/48

(57) ABSTRACT

A device for the observation, imaging and uninterrupted culturing of embryos and cells, the device comprising: an incubator; a mixing box located above the incubator; gas sensors located in the mixing box; solenoid valves attached to the mixing box; a first clear panel located on the top of the incubator, and configured to allow one to see into the incubator; a first light source located above the first clear panel, and the first light source configured to provide light into the incubator; a door attached to the incubator, the door configured to provide access to the inside of the incubator, and the door located below the access door; a rotatable platter located below the incubator, the rotatable platter comprising: a first insert configured to removably sit on the platter; a first culture dish configured to removably sit in the first insert; a second insert configured to removably sit on the platter; a second culture dish configured to removably sit in the second insert; a first moveable camera located under the rotatable platter, and configured to view a culture dish located generally directly above the first moveable camera, and the first moveable camera generally in alignment with the first light source; a computer in communication with the first moveable camera, rotatable platter, sensors, solenoid valves, touchscreen display, and light source. A device for the observation, imaging and uninterrupted culturing of embryos and cells, the device comprising: a top portion, the top portion comprising: a housing; a horizontal surface on the housing; an angled surface abutting the horizontal surface; an access door located generally on the housing; a first heating surface located on the horizontal surface; a touch screen display located on the angled surface; a middle portion abutting the top portion; the middle portion comprising: an incubator; a mixing box located above the incubator; gas sensors located in the mixing box; solenoid valves attached to the mixing box; a first clear panel located on the top of the incubator, and configured to allow one to see into the incubator; a first light source located above the first clear panel, and the first light source configured to provide light into the incubator; a door attached to the incubator, the door configured to provide access to the inside of the incubator, and the door located below the access door; a bottom portion abutting the middle portion, the bottom portion comprising: a rotatable platter, the rotatable platter comprising: a first insert configured to removably sit on the platter; a first culture dish configured to removably sit in the first insert; a second insert configured to removably sit on the platter; a second culture dish configured to removably sit in the second insert; a first moveable camera located under the rotatable platter, and configured to view a culture dish located generally directly above the first moveable camera, and the first moveable camera generally in alignment with the first light source; a computer in communication with the camera, rotatable platter, sensors, solenoid valves, touchscreen display, and light source.

### TECHNICAL FIELD

This invention relates to a device which may be used for providing a controlled environment for the uninterrupted observation and optical comparisons of the stages of development, culturing, maturing levels at various relevant timed intervals.

### BACKGROUND

Currently, there are several devices, which provide an incubated environment and or have the ability to take pictures of embryos and send this information to a user.

The problem facing the current units is that they do not provide all the components necessary to give the embryos the best chance to survive and be the best for preimplantation. These systems are cumbersome, have a large amount of service requirements and are not consistent in their performance.

An additional problem faced by the conventional devices is an inferior or inadequate gas providing system for monitoring and maintain the desired of levels CO2, N2 and O2 and a filtering system to provide continual clean air within these environments. The current devices may not have concise methods for monitoring the gases within the device and therefore not provide the best level of gases. They may also lack in the filter technology necessary to keep the air quality acceptable in the environment.

One problem facing the current units that may take images is that each holding system will only accommodate one type of dish or holding device slide provided by the manufacturers. Therefore the user may only use one dish, which may not be the best dish, and may only allow one type of usage.

An additional problem facing the current units is that when accessing a dish from their holding device, all the dishes in the unit may be exposed to the outside environment and the environment of the laboratory. Therefore if the holding device holds six dishes each time one dish is accessed all six dishes would be exposed to contaminants in the laboratory environment.

An additional problems facing current units is that the dishes may be very large and when arranged in a linear sequence, it requires a very large footprint.

And additional problems facing the current units is that most contain only one camera system, and if the camera system fails, the unit would become inoperative.

Most known systems only have one camera. If manufacturers of devices wanted to include two cameras, this would require a major overhaul to the device.

Thus there is a need for a device for observation, imaging and uninterrupted culturing of embryos and cells that overcomes the above listed and other disadvantages.

### SUMMARY OF THE INVENTION

The invention relates to a device for the observation, imaging and uninterrupted culturing of embryos and cells, the device comprising: an incubator; a mixing box located above the incubator; gas sensors located in the mixing box; solenoid valves attached to the mixing box; a first clear panel located on the top of the incubator, and configured to allow one to see into the incubator; a first light source located above the first clear panel, and the first light source configured to provide light into the incubator; a door attached to the incubator, the door configured to provide access to the inside of the incubator, and the door located below the access door; a rotatable platter located below the incubator, the rotatable platter comprising: a first insert configured to removably sit on the platter; a first culture dish configured to removably sit in the first insert; a second insert configured to removably sit on the platter; a second culture dish configured to removably sit in the second insert; a first moveable camera located under the rotatable platter, and configured to view a culture dish located generally directly above the first moveable camera, and the first moveable camera generally in alignment with the first light source; a computer in communication with the first moveable camera, rotatable platter, sensors, solenoid valves, touchscreen display, and light source.

The invention also relates to a device for the observation, imaging and uninterrupted culturing of embryos and cells, the device comprising: a top portion, the top portion comprising: a housing; a horizontal surface on the housing; an angled surface abutting the horizontal surface; an access door located generally on the housing; a first heating surface located on the horizontal surface; a touch screen display located on the angled surface; a middle portion abutting the top portion; the middle portion comprising: an incubator; a mixing box located above the incubator; gas sensors located in the mixing box; solenoid valves attached to the mixing box; a first clear panel located on the top of the incubator, and configured to allow one to see into the incubator; a first light source located above the first clear panel, and the first light source configured to provide light into the incubator; a door attached to the incubator, the door configured to provide access to the inside of the incubator, and the door located below the access door; a bottom portion abutting the middle portion, the bottom portion comprising: a rotatable platter, the rotatable platter comprising: a first insert configured to removably sit on the platter; a first culture dish configured to removably sit in the first insert; a second insert configured to removably sit on the platter; a second culture dish configured to removably sit in the second insert; a first moveable camera located under the rotatable platter, and configured to view a culture dish located generally directly above the first moveable camera, and the first moveable camera generally in alignment with the first light source; a computer in communication with the camera, rotatable platter, sensors, solenoid valves, touchscreen display, and light source.

### BRIEF DESCRIPTION OF THE DRAWINGS

The present disclosure will be better understood by those skilled in the pertinent art by referencing the accompanying drawings, where like elements are numbered alike in the several figures, in which:

FIG. 1 is an illustration of the device for observation, imaging and uninterrupted culturing of embryos;

FIG. 2 is an exploded view of the device from FIG. 1;

FIG. 3 is a sectional side view of the device from FIG. 1;

FIG. 4 is a perspective view of the middle portion of the device;

FIG. 5 is a top view of the rotatable platter;

FIG. 6 is a top view of one embodiment of the insert;

FIG. 7 is a top view of another embodiment of the insert;

FIG. 8 is a top view of still another embodiment of the insert; and

FIG. 9 is a top view of a fourth embodiment of the insert.

### DETAILED DESCRIPTION

The device consists of an incubated portion to control the environment for maintaining proper levels of Carbon Dioxide (CO2), Nitrogen (N2) and Oxygen (O2), a camera system, a rotatable platter for placing and hold culturing dishes and rotating system for the platter. The device includes a recirculating gas and air system to repeatedly adjust the gas levels for better embryo development. The device includes an air filtration system consisting of HEPA filters, carbon and may include potassium permanganate for removing particulates and volatile organic compounds in the gas and air. The invention has a unique system of interchangeable dish holding devices for multiple dishes, whereas they may hold multiple and various wells designs and configurations, moving in a rotational configuration. Multiple types and sizes of dishes may be used allowing the invention to accommodate more procedural types and needs at the same time. Dishes in their designated holding devices will allow a camera to take images of the specimens, from underneath or from above at desired time intervals. These dishes may also be etched and or coded to allow the camera system to identify each individual dish. The invention has the capability to contain more than two camera systems, which may operate together or independently of each other. The invention will include probes and sensors to report and validate the performance of the ongoing systems, such as VOC metering, Ph. measuring devices, mouse embryo assay testing capabilities and particulate counters. This invention will help maintain acceptable levels of air quality, improved performance, and to assure the user that the conditions are optimal for embryo culture or for the designed results of the user. The invention will provide the user with images of each embryo or specimen, at predetermined time intervals to provide the user with a better understanding of the development of each embryo or specimen, to give the user a better idea of which embryos may be the better embryo for implantation, cryopreservation or such. The invention provides a complete environment to culture the embryos for a period from the first day through blastocyst or implantation into the patients, which in most cases are approximately 5 or 6 days. The invention will overall provide a superior culturing system, along with information, and images that will ultimately allow for better embryo selection, embryo implantation and a better chance of a live birth

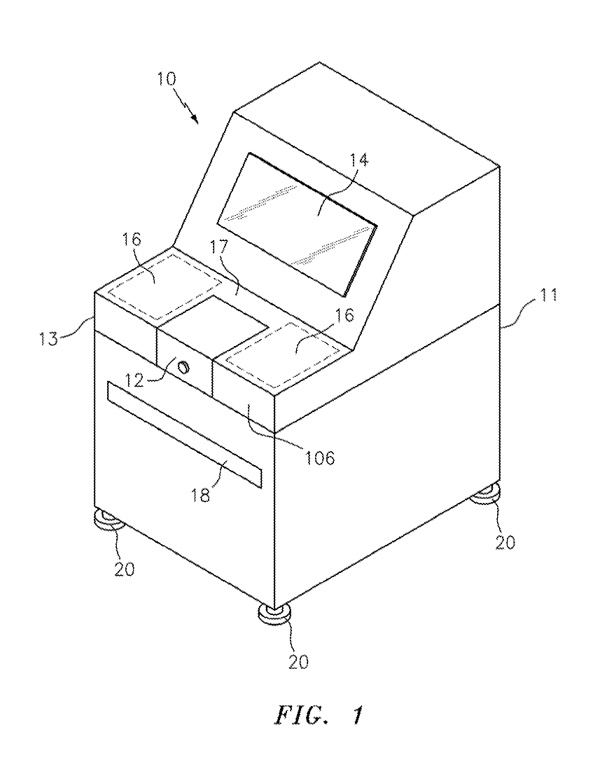

FIG. 1 shows a perspective view of one embodiment of the device 10. The device comprises a base unit 11, an access door 12 located on the front 13 of the base, a touchscreen 14 on the front 13 of the base 11, at least on one heated surface 16 on a flat and generally horizontal surface 17 of the device, and a pull out drawer 18 on the front 13 of the base 11. The drawer 18 may be used for a keyboard or touchscreen. The device 10 may have four feet 20 for leveling the device 10; these feet 20 may be configured to minimize vibration to the device 10. The device may be configured and sized such that it may be sit on a desk, a floor, or on a vibration-eliminating table.

The access door 12 allows the user to access the inside of the device 10, which provides access to the incubator, not shown in this figure. In addition, an incubated system, not shown in this figure, will also be within the base unit 11. The access door 12 may be opaque or clear depending on the needs of the user. In one embodiment, there may be two heated surfaces 16, to the left and the right of the access door 12. These heated surfaces 16 may allow the user to temporarily place dishes containing the specimens or embryos for a brief period of time. The heated surfaces will keep the dishes and the embryos from losing heat.

The touchscreen 14 will be in communication with a computer that controls the device 10. Throughout the following discussion, numerous references will be made regarding servers, services, engines, modules, interfaces, portals, platforms, or other systems formed from computing devices. It should be appreciated that the use of such terms are deemed to represent one or more computing devices having at least one processor configured to or programmed to execute software instructions stored on a computer readable tangible, non-transitory medium. For example, a server can include one or more computers operating as a web server, database server, or other type of computer server in a manner to fulfill described roles, responsibilities, or functions. Within the context of this document, the disclosed computers are also deemed to comprise computing devices having a processor and a non-transitory memory storing instructions executable by the processor that cause the device to control, manage, or otherwise manipulate the features of the assemblies. The touchscreen 14 will allow a user to program the systems, display images taken by the internal camera systems, allow the user to program the timing of these images, control the gas mixture within the device, and to display any information the user wishes to see at any particular time.

The access drawer 18 will allow a keyboard to be placed on it and other electronic devices, which may be used to interface with the device and/or computer.

The device 10 may have a fairly compact size of about 18 inches to about 24 inches wide, about 18 inches to about 24 inches deep, and up to about 36 inches tall. This will allow the device 10 to be small enough to be placed on a tabletop for usage, or an anti-vibration table.

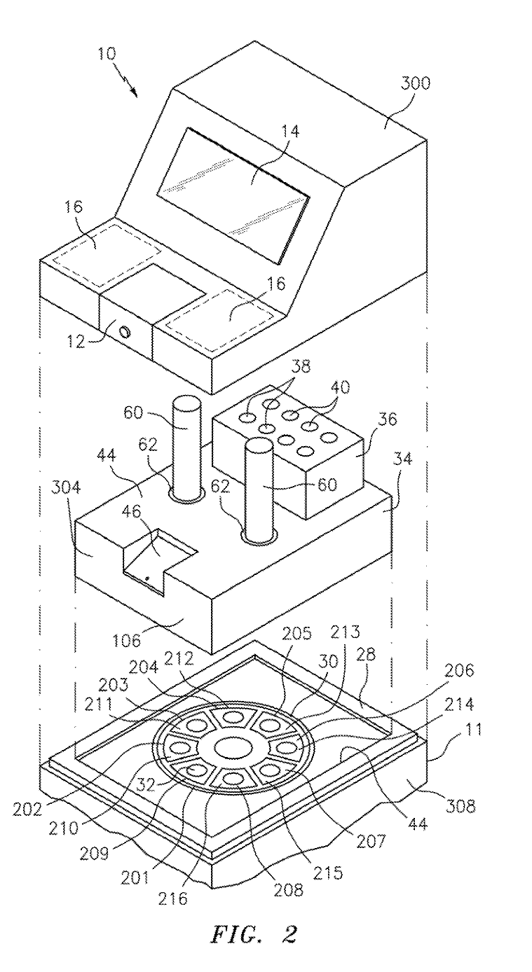

FIG. 2 is an exploded view of the device 10. The base 11 is shown comprising a deck 28; a rotatable platter 30, the rotatable plater holds embryo culture dishes 32. The embryo culture dishes are to be observed and imaged. In this particular embodiment there may be eight sections 201, 202, 203, 204, 205, 206, 207, and 208. Each section 201-298 may comprise an individual insert 209, 210, 211, 221, 213, 214, 215, 216. Each insert 209-216 may be interchangeable, securely attached to this rotational platter and will hold the dishes 32 in place for the imaging done by the camera.

In this embodiment, the rotational platter 30 may be placed in the incubator located in the incubator housing 34, which will maintain the proper mixture of gases for proper embryonic growth. The incubator housing 34 will be configured to provide a proper mix of CO2, N2 and O2. In this embodiment a gas mixing box 36 is in fluid communication with the incubator system. The gas mixing box 36 is used to monitor and regulate the gases within the incubator housing 34. The gas mixing box 36 comprises a plurality of sensors 38. The sensors are configured to monitor the CO2, N2 O2, and temperature within the incubator housing 34. The mixing box 36 comprises a plurality of solenoids valves 40. A computer will control the solenoid valves 40 such that the solenoid valves 40 will allow the introduction of the specific gases CO2, N2 and O2 according to readings from the sensors 38.

The incubator housing 34, includes two light sources 60 which will project light into the incubator housing 34 through two clear panels 62 made of clear glass or a polystyrene or any other suitable generally transparent material, which will allow the light to pass through to the dish 32, illuminating the sample, allowing a camera, not shown, to record images from the dish 32. This camera is placed below the deck and therefore takes inverted images of the embryo in the dishes 32.

The incubator housing 34 is attached to outside gas sources, such as CO2, N2, and O2 from incoming gas lines. The gas sources may be laboratory gas tanks.

The top portion 300 of the device 10 comprises the access door 12, touchscreen 14 and the two heated surface areas 16. The middle portion 304 of the device 10 comprise the light sources 60, gas mixing box 36, and sensors 38 and solenoid valves 40.

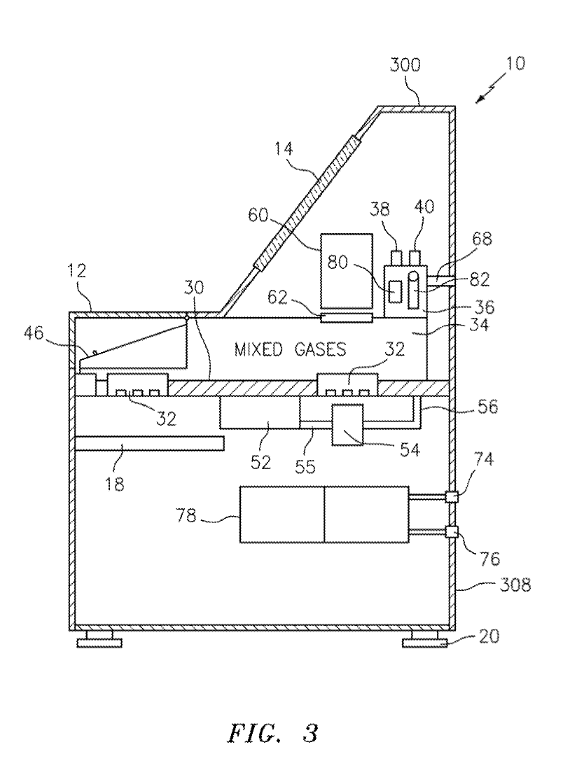

The bottom portion 308 of the device 10 may comprise two cameras (shown in FIG. 3) underneath the rotatable platter 30 for the imaging of the specimens in the dishes 32. The two camera system allow these cameras to take images at the same time or independently, which may cut down on the amount of movement of each of the cameras but will also allow the users to integrate two different types of cameras. An example of this would be if one camera simply takes black and white images, while the second camera takes colored images and/or 3-D images.

Having two cameras will allow the end-users to maintain a preferred camera, such as a simple black and white imaging system, and then to use or review additional camera images or photographic techniques to obtain additional information when desired or necessary. This two camera setup may cause less disruption to the samples in the dishes 32.

FIG. 2 also shows an access door 12, which is directly above a door 46. The door 46 may be clear or opaque. The clear door 12 is part of the incubator housing 34, and therefore allows a user to view which dish 32 is below the door 12, before opening the clear door and exposing that dish to the environment. The use of this door configuration allows only one dish 32 to be exposed to the room environment, as the other dishes 32 not exposed to the ambient environment but are kept within the incubator housing 34 environment.

FIG. 3 shows a side cross-sectional view of the device 10. The top portion 300 comprises the touchscreen 14 and access door 12. Illustrated is the incubator housing 34, which when in operation contains the mixed gases, which is controlled by the mixing box 36, solenoids 40 and sensors 38. Attached to the mixing box 36 are incoming gas lines 68. In one embodiment, there may be a dedicated incoming gas line 68 for each of the necessary gases: CO2, N2 and O2. The gas lines 68 are in communication with a gas supply generally located outside of the device 10. A transparent door 46 is located generally in the front portion of the incubator housing 34. The transparent door 46 allows access to the interior of the incubator housing 34. Beneath the transparent door 46 will lay an embryo culture dish 32. Thus, the transparent door 46 will provide access to a user to remove, replace, or check on the dish 32 located below the door 46.

A plurality of dishes can be set on the rotatable platter 30. The rotational platter 30 may be rotated by a motor 52. The motor 52 will be controlled by a computer 78.

In the rear portion of the device 10 are the light sources 60. the light sources will sit above the incubator housing 34 and allow light to pass through this incubator, by way of an airtight window 62 made of glass, polycarbonates, or any other suitable material. The light source 60 will provide light to illuminate the dish 32 located beneath the light 60. Below the rotatable platter 30 is a camera 54, which is mounted on a XYZ camera moving system 55, which will allow the camera to move in three dimensions and take images throughout the surface of the dish 32. The camera 54 may be controlled by the computer 78 or a user to view the embryonic specimens, which are held within wells of the dishes 32 and are in position to be imaged by the camera 54. The cameras 54 will be supported by under mounted supports 56 which will allow them to maintain image clarity and accuracy. The dishes 32 may be held in place by inserts located in the rotatable platter 30 (inserts not shown), in order to align wells of the dishes 32 with the camera 54 below.

The mixing of the gases CO2, N2 and O2 may be performed in the mixing box 36 in a continual flow of circulating the gases between the incubator and the mixer box 36. The incoming gases through the incoming gas lines 68 will enter the mixing box 36 where they will be monitored by the sensors 38 and will be adjusted through the use of the solenoid valves 40 to allow the precise amount of each gas to enter the mixing box 36 and adjust to the proper overall gas concentration. The mixing box 36 is in fluid communication with a pump 80 and a filter 82. The pump 80 will move the gases throughout the system and the filter 82 will filter the gas for particulates and volatile organic compounds. The pump 80, filter 82, and mixing box 36 comprise a recirculating gas system. The recirculating gas system is important to maintaining the proper mixture of gases, and also to protect the embryos from particulates and volatile organic compounds.

Located in the bottom portion 308 of the device are electrical connectors 74, and Internet and other communication connectors 76, along with the computer 78 which will be used to operate the system. The computer 78 is in communication with the touch screen 14 which allows for user control of the device 10.

The embodiment shown in FIG. 3, allows for an ergonomic advantage for the users in that the use of a rotatable platter 30 for holding the dishes 32 allows a reduced size and footprint of the platter 30 as well as size and footprint of the device 10 itself. The positioning of the mixing box 36 and the light sources 60 to the rear of the device 10, allow them to be concealed within the housing of the top portion 300 of the device 10, which then allows the touchscreen 14 to be more ergonomically accessible and at an ergonomic working angle and at a good working height for the user.

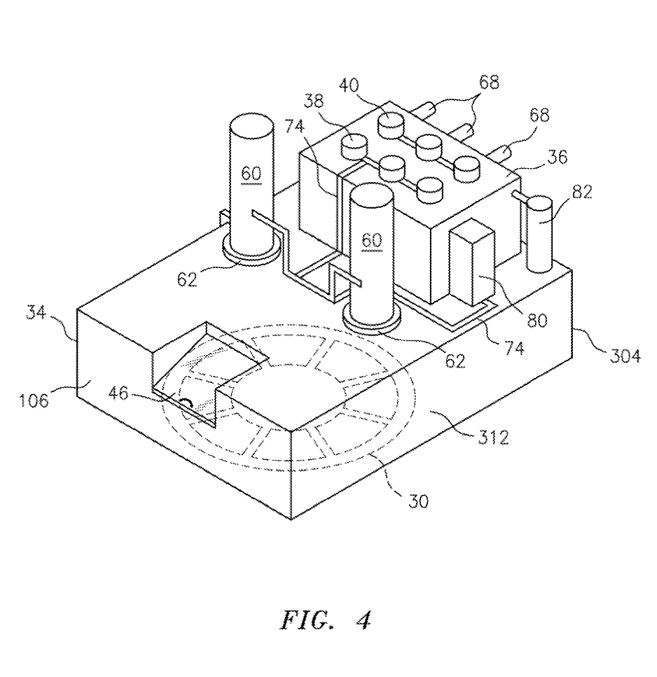

FIG. 4 is a perspective view of the middle portion 304 of the device 10. Shown is the incubator housing 34, the incubator 312 of the device 10 is used to provide a controlled gas environment as well as to control the temperature of the area of which the culture dishes 32 will reside in. The incubator housing 34 consists of the incubator 312 for containing the gases within. The incubator 312 may be about 18 inches to about 25 inches wide, about 18 inches to about 25 inches deep, and about 3 inches to about 5 inches in height. In the front portion of the incubator housing 34 will be an access door 46, which may be clear or opaque, and the door 46 is used to allow access to the interior of the incubator housing 34. When the access door 46 is open the user will have access to an embryo culture dish 32, which resides in the front portion of rotatable platter 30, which is shown with dashed lines in this figure. The incubator housing 34 in this embodiment may have two light sources 60 which will project the light through a window 62, and onto a dish 32 which is sitting on the platter 30, and through to the camera underneath, not show in this view. These light sources 60 may be secured to the device 10 by support brackets, which may be attached to the base portion of the invention or to the incubator system itself. Electrical connectors are in communication with the light sources 60, solenoids 40, sensors, 30, and pump 80. In this embodiment the gas mixing box 36 is attached to rear upper portion of the incubator housing 34, which contains incoming gas lines 68, solenoids 40 for adjusting the incoming gases, and sensors 38 to continuously and/or repeatedly monitor the gases and to transmit the gas levels to the computer 78. The computer controls the solenoids 40 to control the amount of gases to enter the system to keep those gases balanced at the desired level. The gases within the system will be recirculated utilizing a pump 80 and a filter 82, which will continuously filter the gases within the incubator 34.

In the embodiment shown in FIG. 4, it can be seen that the entire platter 30 is inside of the incubator 312. The dishes 32 are held in the platter 30, and the platter has an opening below each dish 32 to allow the camera 54 located below the platter 30 to photograph upward into the dish 32. In other embodiments, the platter 30 may be made out of transparent material to allow the camera 54 to photograph the dish 32 sitting on the platter 30. Since the entire platter 30 is located within the incubator housing 34, every dish 32 on the platter 30 is also in the incubator housing 34 during the embryonic growth, which may be from 1 to 6 days, and to maintain the correct gas mixes, temperature during this time. The window 62 atop the incubator housing 34 allows the light from the light source 60 pass through the incubated airspace through the dish 32, and to the camera 54 located below the platter 30.

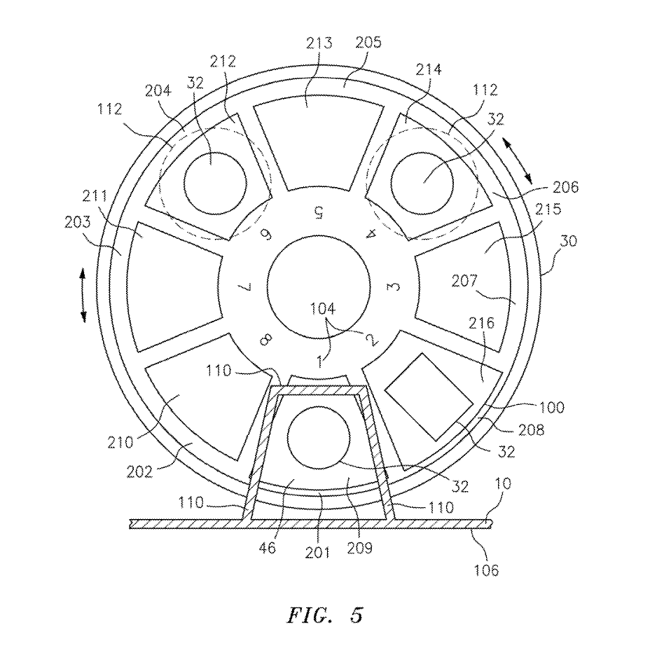

FIG. 5 is a top view of the rotatable platter 30 on which the dishes 32 may be placed during imaging and incubation. This platter 30 is unique in that it rotates in a circular motion, allows eight dishes 32 (eight in this embodiment, may be more or less in other embodiments) to be held on the platter 30 in the incubator housing 34. The platter 30 may be divided into eight sections 201, 202, 203, 204, 205, 206, 207, 208 corresponding to each area configured to hold a dish 32. Within each of these eight sections 201-208, there may be a removable insert 209, 210, 211, 212, 213, 214, 215, 216, which will be interchangeable in this platter 30. The inserts 209-216 will be made and configured to hold several types of dishes which may contain multiple configurations of wells and points of interest within the dish 32. In this embodiment each section 201-208 is numbered, one through eight, to allow the user to identify the insert and dish related to the section 201-208. The computer 78 may be programmed to verify the location of each section with respect to the cameras 54 and door 46.

These inserts 209-216 may accommodate most dish sizes currently used in the marketplace, including such sizes as 60 mm, 38 mm, round dishes as well as square dishes. In FIG. 5, the number one position of the platter 30 is located below the door 46. Each insert 209-216 may include multiple dish configurations. A front wall 106 of the device 10 is shown. The dish 32 located in position 1 is the only dish 32 in the platter 30 that will be exposed to the room environment when the access door 46 is open. The other seven inserts 210-216, and dishes 32 and the specimens sitting within these dishes 32 will remain within the incubated environment. The sealing walls 110 of the device generally prevent contamination of the atmosphere for the other inserts that are not below the door 46 when the door 46 is opened. The circles 112 shown in dashed line represent where the light sources 60, clear panels 62, and cameras 54 generally line up in relationship to the platter 30. In the current rotation orientation as shown in FIG. 5, the light sources 60 and cameras 54 are aligned to the positions 4 and 6 on the platter 30. This invention allows for two dishes 32 at locations 4 and 6 to be views by the camera 54 simultaneously, or at different times. The dual camera 54 set up also allows the invention to include to different types of cameras to accommodate the needs or the size of the end-user. In one example, the camera under position 4 may be a black and white camera and the camera below position 6 may be a color camera, or 3D camera for additional and different type of imaging. In addition to this example, the invention may include additional cameras aligned with some or all of the positions 3, 4, 5, 6 and 7, thus allowing the use of up to 5 cameras in the invention. The rotatable platter 30 is a compact size and has interchangeable inserts to accommodate multiple types of dishes or other holding devices at the same time. The rotation of the platter 30 and operation of the cameras will be controlled by the computer 78.

FIG. 6 shows a close up view of an insert 209. As shown the insert 209 may include one or more locking tabs 124 which will allow the insert 209 to correctly align itself within the particular section 201-208, which is important for proper imaging by the cameras 54. FIG. 6 shows a squared embryo culture dish receiving area 120 located on the insert 209. The receiving area 120 is configured to hold a squared embryo culture dish. The insert 209 may be made of aluminum or stainless steel, or any other suitable material. The insert 209 will be attached to the platter 30 for imaging accuracy as well as to heat the insert and maintain the heat of the dish 120 or other dish 32 which may be placed within the insert. FIG. 6 shows twelve well openings 122 located in the receiving area 120 of the insert 209. These twelve openings in the insert will allow the light to pass through the dish and allow the images to be captured by the camera below the dish. The wells of the dish will concisely fit into the well openings 122 of the insert, thereby allowing the light to only illuminate these wells and those portions of the dish and to allow the camera to take images. The receiving area 120 may also have receiving slots 125 to configured to receive tabs molded within the squared embryo culture dish, to firmly hold the dish within the receiving area 120.

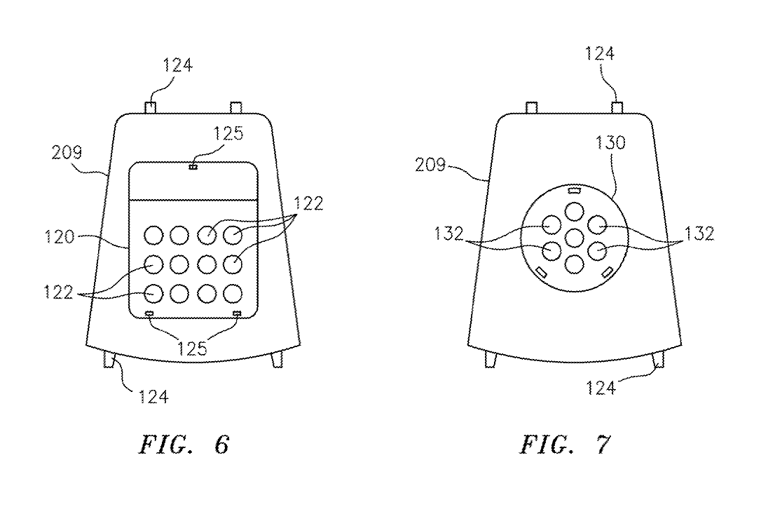

FIG. 7 shows a second embodiment of an insert 209, its tabs 124 and a round embryo culture dish receiving area 130. The receiving area has seven well openings 132 which are configured to line up with the wells of a round embryo culture dish. The openings 132 allow light from the light sources 60 to puss through the round embryo culture dish, through the openings 132, and to the camera 54 located below the platter 30.

FIGS. 6 and 7 illustrate the versatility of the inserts 209-216, and their ability to accommodate multiple sizes and shapes of embryo culture dishes for the imaging and recording of the specimens in the dishes over time. The squared embryo culture dish to be used in FIG. 6 may be about 50 mm wide by about 16 mm in length. The round embryo culture dish to be used in FIG. 7 may have a diameter of about 38 mm. The receiving areas 120 and 130 are configured to have a precise fit with their respective embryo culture dishes, which allows the incubator to remain sealed while allowing images to be taken.

FIG. 8 shows an insert 209 which can accommodate an about 60 mm embryo culture dish which has a clear flat bottom and no wells. The dish holding area 150 of the insert 209 is configured to hold the entire dish, the dish holding area 150 has an opening 151 that allows light from the light sources 60 to travel through the embryo culture dish, through to the camera 54 below the platter 30. When using such a clear flat bottom dish, the user will generally place embryos in droplets in the dish; the droplets may be located anywhere in the flat bottom area of the dish. FIG. 8 shows where the droplets 152 may be located in one example. The camera 54 along with recognition software located in the computer 78 will individually locate the droplets 152 and then the embryos within these droplets in order to take images of these embryos. This feature will be an aided by the XYZ camera moving system 55.

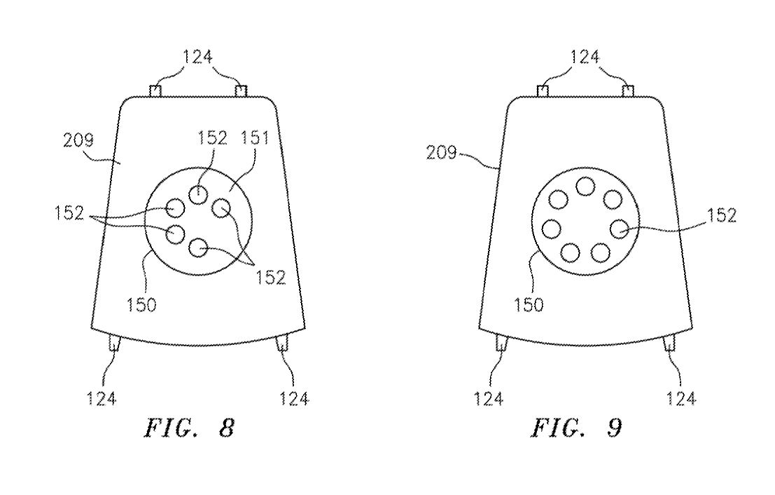

FIG. 9 shows an insert 209 with a 38 mm clear, flat bottom, generic culture dish receiving area 150. Randomly placed droplets 152 will be located on the flat bottom of the dish. The camera 54 along with recognition software located in the computer 78 will individually locate the droplets 152 and then the embryos within these droplets in order to take images of these embryos. This feature will be an aided by the XYZ camera moving system 55.

This invention has many advantages. This invention relates to an improved embryo culturing device, embryo maturation, imaging and the observation of the embryos, to provide enhanced imaging and to allow a more concise method for selecting the embryo for implantation and/or cryopreservation. The invention contains a temperature controlled incubated environmental chamber, which can maintain the internal temperature of about 37 degrees Celsius; and maintains proper gas levels for CO2, and N2 and O2 will consistently purifying the air through a recirculating gas system, filters, and a display touchscreen for programming and monitoring the system. The display screen will display key features and information related to the ongoing system, such as the internal temperature of the device, gas levels, such as CO2, N2 and oxygen and images. The display screen will also be used to show each embryo or specimen in the sequence selected by the user. The embryos will be shown at specifically selected time intervals, metabolic stages and at any other times or stages selected by the user. The system will allow the user to compare past and present embryos of the patient, the program and the clinic. The invention consists of a unique rotational platter system, which will allow the concise imaging of embryos and allows the use of multiple types of dishes, while maintaining a small footprint and overall size of the invention. This rotational platter will reside in the incubated environment of the invention while allowing the images to be taken threw a lighting and camera system. The rotational platter system allows all the dishes to be placed and removed from invention, from within the incubator system, from the frontal portion. This rotational configuration allows one dish to be viewed and retrieved, thereby allowing the additional dishes to remain within the incubator and the mixed gas environment, and therefore only exposing one dish to the room environment. This feature therefore allows the remaining dishes to the better temperature and environmentally control. The invention is has a unique advantage over the competition is that it can incorporate several patented devices and combining patented technologies into a far more effective and reliable system for the culturing of embryos, which are the property of the assignee and the inventors. The combination of these patents along with addition of unique key features within this invention, establishing a stand-alone, more complete incubation and maturation device for the purpose of long-term uninterrupted embryo culture. The invention includes an interchangeable dish holding insert, which is to be inserted into the platter assembly and to be removed and replaced to match particulate dishes or holding device to be used. The platter will be placed along with or atop the deck, which will be heated to provide effective heating and to meet the heating needs of the embryos or specimens. An additional advantage of the invention is it allows the collection of images at key intervals of development. Imaging will be taken and collected to be used in a determined sequence of images for the evaluation by the user or embryologist. This timed sequencing and maturation sequential will be used to monitor the growth levels and the development of the embryos. The invention will allow the user to observe and annotate and describing the maturation of a human embryo. This will also allow the comparison of these images to the prior images of same embryos, other embryos of the patient, and embryos from past successful treatments for this clinic or laboratory. The images will be sequenced in a to collection of the images in a useful collection for comparisons. These sequences and comparisons of those in times, morphological development and other sequences desired by the user to determine which embryos to be used in implantation or cryopreservation. The invention will take the images at planned and timely intervals, which may be at intervals of any combination of minutes or hours. The intervals of the images may be adjustable by the user to the frequency they find most effective for their protocols, practice and clinic. The culture media to be used in the invention will be consistent with the media solution used for extended and uninterrupted embryo development. The embryos will be matured from the retrieval, referred to as day “0”; they then will be cultured through to blastocyst development or Day 5 or 6. These embryos have a better chance of implantation and eventual live birth, after they have been matured to the blastocyst stage. This media is consistent with the current media products, trade named Global®, which is a proprietary media solution of the assignee. The invention will allow the use of a multiple number of medium solutions and protocols. The invention incorporates a system to maintain the temperature of the embryos. The system includes a heating system for the deck, platter and incubator as well as temperature reading probes throughout the invention to assure that the temperature are what is need and what is being reported. There are controllers over the incoming gas ports for the control of the CO2, N2 and O2 levels, injector ports and control mechanisms. The invention may include its own gas mixing system and sensors for adjusting the gas levels as needed for the correct gas balance within the incubator portion. The invention is a combination of the described incubator filters and may include at least one motor or pump in the device to provide consistent airflows, air velocities and air exchanges. Currently, most systems utilize a single dish or holding configuration for holding the embryos during the process. The invention includes a unique feature, which will allow the user to select the dish type they want to use, using multiple insert and dish hole configurations and the system will provide chooses to recognize the different dish configurations and set up the positioning for the images to be taken for the configuration of each particular dish in the rotational platter configuration. This feature is important to the invention in that it allows the dishes used to be specific for the different needs of the user. To illustrate this embodiments, in one example a dish may contain many smaller wells which may contain less amounts of culture media, of approximately 100 microliters of media in the well. A second user may desire to use more media, of up to 300 ultra liters per well, which would require larger wells, which may position the wells differently in each dish configuration. The device can provide an improved device for the long-term uninterrupted maturation of embryos through to the stage of development desired by the user providing the proper amount of CO2, N2 and oxygen. The device can provide a precise sequence of images of the embryos during development, which will allow the embryologist to better select a viable embryo. The device can provide a favorable clear environment by including an effective air filtration system to remove particulates and VOC's from the incubated environment. The device can provide an improved device for the long-term uninterrupted maturation of embryos, which contains a real time monitoring system for the key indices such as ph., levels, CO2, N2, air quality and other key items. The device can provide a single device which may be used separately to mature embryos, which will include all the featured needed, such as an incubator section, gas input and the ability to take images at desired times. It would be highly desirable to provide a device, which will contain a controlled environment of temperature and gas levels, along with a uniquely designed dish to hold the embryo in place for imaging, the ability to take images at designated and at specific time intervals, and to accomplish this with a rotational holding deck. It would be highly desirable to provide a device, which will contain a controlled environment of temperature and gas levels. A uniquely designed dish to hold the embryo in place for imaging, images taken at designed and specific time intervals. To utilize an embryo culture media which will allow the uninterrupted culturing of the specimen, an air filtration device, which will clear the enclosed environment with greater reduction of particulate, VOC's and CAC's.

It should be noted that the terms “first”, “second”, and “third”, and the like may be used herein to modify elements performing similar and/or analogous functions. These modifiers do not imply a spatial, sequential, or hierarchical order to the modified elements unless specifically stated.

While the disclosure has been described with reference to several embodiments, it will be understood by those skilled in the art that various changes may be made and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure not be limited to the particular embodiments disclosed as the best mode contemplated for carrying out this disclosure, but that the disclosure will include all embodiments falling within the scope of the appended claims.

What is claimed is:

1. A device for the observation, imaging and uninterrupted culturing of embryos and cells, the device comprising:
an incubator;
a mixing box located above the incubator;
gas sensors located in the mixing box;
solenoid valves attached to the mixing box;
a first clear panel located on the top of the incubator, and configured to allow one to see into the incubator;
a first light source located above the first clear panel, and the first light source configured to provide light into the incubator;
a door attached to the incubator, the door configured to provide access to the inside of the incubator, and the door located below the access door;
a rotatable platter located below the incubator, the rotatable platter comprising:
a first insert configured to removably sit on the platter;
a first culture dish configured to removably sit in the first insert;
a second insert configured to removably sit on the platter;
a second culture dish configured to removably sit in the second insert;
a first moveable camera located under the rotatable platter, and configured to view a culture dish located generally directly above the first moveable camera, and the first moveable camera generally in alignment with the first light source;
a computer in communication with the first moveable camera, rotatable platter, sensors, solenoid valves, touchscreen display, and light source.

2. The device of claim 1 further comprising:
a heater located in the incubator and in communication with the computer.

3. The device of claim 1 further comprising:
a second clear panel located on the top of the incubator, and configured to allow one to see into the incubator;
a second light source located above the second clear panel, and the second light source configured to provide light into the incubator;
a second moveable camera located under the rotatable platter, and configured to view a culture dish located generally directly above the second moveable camera, and the second moveable camera generally in alignment with the second light source.

4. The device of claim 1, wherein when the door is opened, only the one insert and the one culture dish directly adjacent to the door is exposed to the ambient atmosphere in a space where the device is located.

5. The device of claim 1, wherein the rotatable platter further comprises:
a third insert configured to removably sit on the platter, the third insert located adjacent to the second insert;
a third culture dish configured to removably sit in the third insert;
a fourth insert configured to removably sit on the platter, the fourth insert located adjacent to the third insert;
a fourth culture dish configured to removably sit in the fourth insert;
a fifth insert configured to removably sit on the platter, the fifth insert located adjacent to the fourth insert;
a fifth culture dish configured to removably sit in the fifth insert;
a sixth insert configured to removably sit on the platter, the sixth insert located adjacent to the fifth insert;
a sixth culture dish configured to removably sit in the sixth insert;
a seventh insert configured to removably sit on the platter, the seventh insert located adjacent to the sixth insert;
a seventh culture dish configured to removably sit in the seventh insert;
an eighth insert configured to removably sit on the platter, the eighth insert located adjacent to the seventh insert;
an eighth culture dish configured to removably sit in the eighth insert.

6. The device of claim 1, wherein the first insert is configured to accept a culture dish selected from the group consisting of 60 mm diameter round dishes, 38 mm diameter round dishes, squared culture dish 50 mm by 16 mm, a squared culture dish with 12 wells; a round culture dish with 7 wells.

7. The device of claim 3, wherein the second moveable camera is a 3d imaging camera.

8. The device of claim 1, further comprising a heatable surface located on the horizontal surface.

9. The device of claim 1, further comprising:
a first solenoid valve;
a second solenoid valve;
a third solenoid valve;
an N2 supple in fluid communication with the first solenoid valve;
an O2 supply in fluid communication with the second solenoid valve; and
a CO2 supply in fluid communication with the third solenoid valve.

10. A device for the observation, imaging and uninterrupted culturing of embryos and cells, the device comprising:
a top portion, the top portion comprising:
a housing;
a horizontal surface on the housing;
an angled surface abutting the horizontal surface;
an access door located generally on the housing;
a first heating surface located on the horizontal surface;
a touch screen display located on the angled surface;
a middle portion abutting the top portion; the middle portion comprising:
an incubator;
a mixing box located above the incubator;
gas sensors located in the mixing box;
solenoid valves attached to the mixing box;
a first clear panel located on the top of the incubator, and configured to allow one to see into the incubator;
a first light source located above the first clear panel, and the first light source configured to provide light into the incubator;
a door attached to the incubator, the door configured to provide access to the inside of the incubator, and the door located below the access door;
a bottom portion abutting the middle portion, the bottom portion comprising:
a rotatable platter, the rotatable platter comprising:
a first insert configured to removably sit on the platter;
a first culture dish configured to removably sit in the first insert;
a second insert configured to removably sit on the platter;
a second culture dish configured to removably sit in the second insert;
a first moveable camera located under the rotatable platter, and configured to view a culture dish located generally directly above the first moveable camera, and the first moveable camera generally in alignment with the first light source;
a computer in communication with the camera, rotatable platter, sensors, solenoid valves, touchscreen display, and light source.
